(19) United States
(12) Patent Application Publication
(10) Pub. No.: US 2019/0069698 A1
(43) Pub. Date: Mar. 7, 2019

(54) PILLOW

(71) Applicant: ODERBAU FURNITURE CO., LTD., Taoyuan City (TW)

(72) Inventor: Ching-Hui Lin, TAOYUAN CITY (TW)

(21) Appl. No.: 15/697,424

(22) Filed: Sep. 6, 2017

(51) Int. Cl. A47G 9/10; A47C 20/02; A47C 27/08

(52) U.S. Cl. CPC A47G 9/1081; A47G 9/1009; A61F 5/56; A47C 27/08; A47C 20/02

(57) ABSTRACT

A pillow is of a dual-layer structure composed of a relatively soft upper main body and a relatively hard main body. The main body is centrally provided with a central unit that is flanked by two lateral units. The central unit has a raised portion for propping up a user's cervical vertebra so as to make the user's head lean backward, thereby keeping his trachea opening and preventing snoring. Each of the lateral units has a lateral depressed portion. When a user sleeps on his side with his head placed on the pillow, the depressed portion prevents his ear from being pressed and serves to lead his head lean backward. Each of the lateral units has its front edge recessed and provided with an inward inclined portion, for receiving the user's shoulder and preventing discomfort caused by pressure. The pillow thus provides comprehensive support to the user's head.

### BACKGROUND OF THE INVENTION

### 1. Technical Field

The present invention relates to pillows, and more particularly to a pillow that helps to improve sleep quality.

### 2. Description of Related Art

Many are harassed with insomnolence, and one major fact that causes insomnolence is an unsuitable sleep pillow. During sleep, one can naturally change his posture several or even more than ten times between sleep-on-back and sleep-on-side positions as a physiologic reaction for health, so as to prevent any part of his body from being pressed long and feeling unwell and sore. In addition, when a man sleeps on his back, the interval between his neck and the bed's surface is different from that when he sleeps on his side. In particular, the sleep-on-back position makes the neck relatively close to the bed surface, so the proper height of the sleep pillow should be relative small. On the other hand, the sleep-on-side position makes the neck and the head relatively far from the bed surface due to his shoulder, and thus needs the proper height of the sleep pillow to be relatively large, in order to effectively support the user's head and neck.

However, all the existing sleep pillow available on the market are of a flat structure, and thus fail to meet the different support requirements for a user's sleep-on-back and sleep-on-side positions. This causes various physical problems related to poor blood circulation, such as neck sprain, shoulder sprain or soreness, shoulder compression pain, compression paralysis, and even adhesive capsulitis. It is clear that the existing sleep pillows leave the foregoing problems unmet and thus fail to address human physiological needs by enabling good sleep.

### SUMMARY OF THE INVENTION

For addressing the shortcomings of the conventional pillows by fully meeting users' sleep requirements, the primary objective of the present invention is to provide a structurally improved pillow, which can meet a human body's height requirements for both sleep-on-back and sleep-on-side position. Particularly, the pillow has an undulate profile so as to well support its user in any sleep posture.

The primary objective of the present invention is to provide an improved pillow, which has two lateral units designed to such support a sleep-on-side user's head that it makes the user's head slightly lean backward for nearly 15 degrees, thereby preventing snoring, ensuring smooth breath, and in turn improving the user's sleep quality. Moreover, the lateral unit has a height that perfectly supports a sleep-on-side user's head, thereby protecting a sleep-on-side user from neck discomfort, hand numbness and poor blood circulation caused by the user's arm or shoulder compression.

The secondary objective of the present invention is to provide an improved pillow, which well supports a sleep-on-back user's head, and makes the user's head slightly lean backward for nearly 15 degrees, thereby preventing snoring, ensuring smooth breath, and in turn improving the user's sleep quality.

Another objective of the present invention is to provide an improved pillow, whose two lateral units each has its front edge provided with an inward inclined portion. The inward inclined portion serves to receive a shoulder of a user sleeping on his side so as to make the user's shoulder more comfortable at the sleep-on-side position.

Still another important objective of the present invention is that since the pillow has a dual-layer structure, it provides comfortable touch with the relatively soft upper main body, and provides good structural support with the relatively hard main body, so as to effectively improve a user's sleep quality.

To achieve the foregoing objectives, the present invention provides a pillow, comprising a main body, and an upper main body integratedly formed on the top of the main body, wherein the upper main body is made of a relatively soft, nontoxic, flexible material, and the main body below it is made of a relatively hard material.

Preferably, the main body is centrally provided with a central unit, which is flanked by two lateral units.

Preferably, the central unit provided with a raised portion. The raised portion has its front edge adjacent to an inclined portion and has its rear edge provided with a depressed portion. The raised portion is configured to prop up a user's cervical vertebra and makes the user's head lean backward, thereby keeping the user's trachea open and preventing snoring. The inclined portion is configured to receive the user's neck-back joint. The depressed portion serves to hold and position a sleep-on-back user's afterbrain.

Preferably, each of the lateral units is centrally provided with a lateral depressed portion. The lateral depressed portion is depressed so as not to make a sleep-on-side user's ear compressed. The lateral depressed portion is peripherally provided with a lateral frame. The lateral frame is such shaped that it supports the user's head from side and guided the user's head to lean backward for a proper angle. Each of the lateral units has its front edge recessed and provided with an inward inclined portion, which receives and protects the user's shoulder from compressive discomfort.

Preferably, the upper main body is shaped according to the main body, and thus is structurally similar to main body for having an upper central unit and two upper lateral units. The upper central unit includes an upper raised portion, an upper inclined portion, and an upper depressed portion. Each of the upper lateral units is formed with an upper lateral depressed portion, and an upper lateral frame. Each of the upper lateral units has its front edge recessed and provided with an upper inward inclined portion.

### BRIEF DESCRIPTION OF THE DRAWINGS

FIG. 1 is a perspective view of a pillow disclosed in the present invention.

FIG. 2 is an exploded view of the pillow.

FIG. 3 is a prospective cross-sectional view of the pillow.

FIG. 4 is an applied view of the pillow wherein a user is sleeping on his back with his head placed on the pillow.

FIG. 5 is another applied view of the pillow wherein a user is sleeping on his back with his head placed on the pillow.

FIG. 6: is an applied view of the pillow wherein a user is sleeping on his side with his head placed on the pillow.

FIG. 7 is another applied view of the pillow wherein a user is sleeping on his side with his head placed on the pillow.

FIG. 8 is a front view of a pillow disclosed in the present invention having its main body provided with support members at two sides of its bottom.

### DETAILED DESCRIPTION OF THE INVENTION

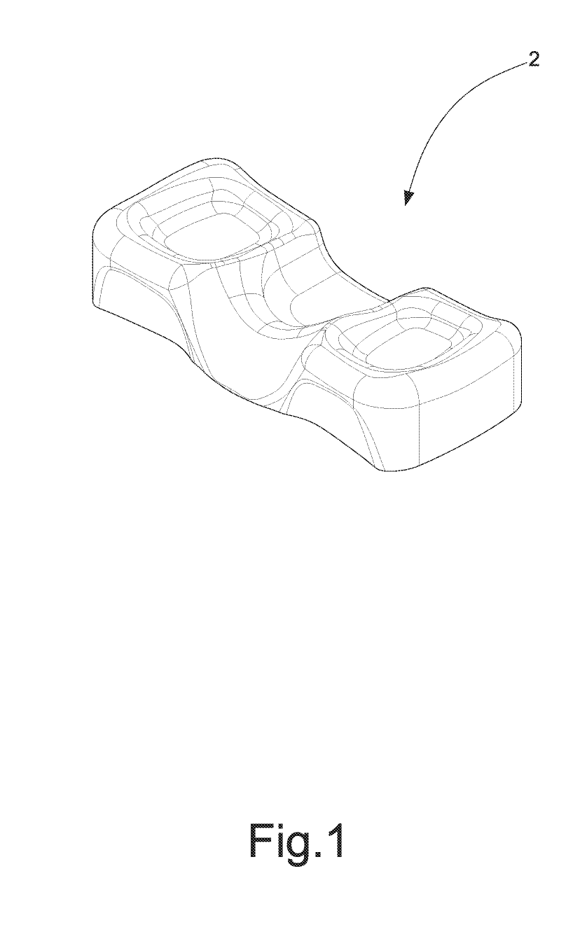

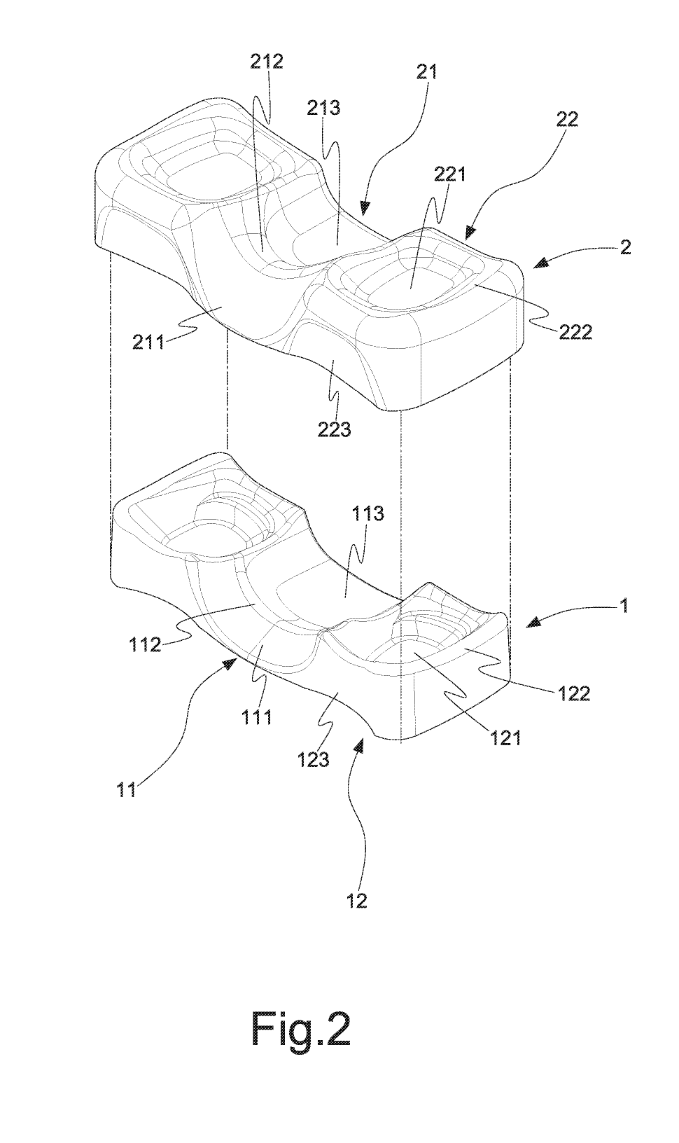

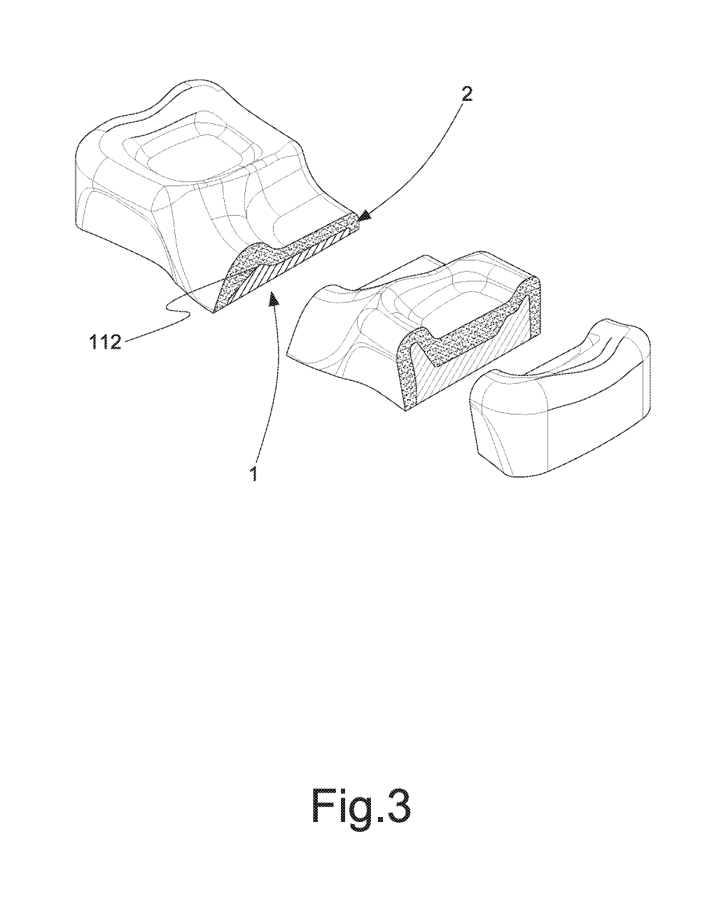

Referring first to FIG. 1, FIG. 2, and FIG. 3, according to the present invention, a pillow comprises a main body 1, and an upper main body 2 provided on the top of the main body 1. The upper main body 2 is made of a relatively soft, nontoxic, flexible material, and the main body 1 below it is made of a relatively hard material.

The main body 1 is centrally provided with a central unit 11, which is flanked by two lateral units 12. The central unit 1 includes a raised portion 112. The raised portion 112 has its front edge adjacent to an inclined portion 111, and has its rear edge provided with a depressed portion 113.

Each of the lateral units 12 is centrally provided with a lateral depressed portion 121. The lateral depressed portion 121 is peripherally provided with a lateral frame 122. The lateral frame 122 is in a predetermined shape. In addition, each of the lateral units 12 has its front edge recessed and provided with an inward inclined portion 123.

The upper main body 2 is shaped according to the main body 1, and thus similar to the configuration of the main body 1, also has an upper central unit 21 and two upper lateral units 22. The upper central unit 21 includes an upper raised portion 212, an upper inclined portion 211, and an upper depressed portion 213. Each of the upper lateral units 22 is formed with an upper lateral depressed portion 221 and an upper lateral frame 222. Each of the upper lateral units 22 has its front edge recessed and provided with an upper inward inclined portion 223.

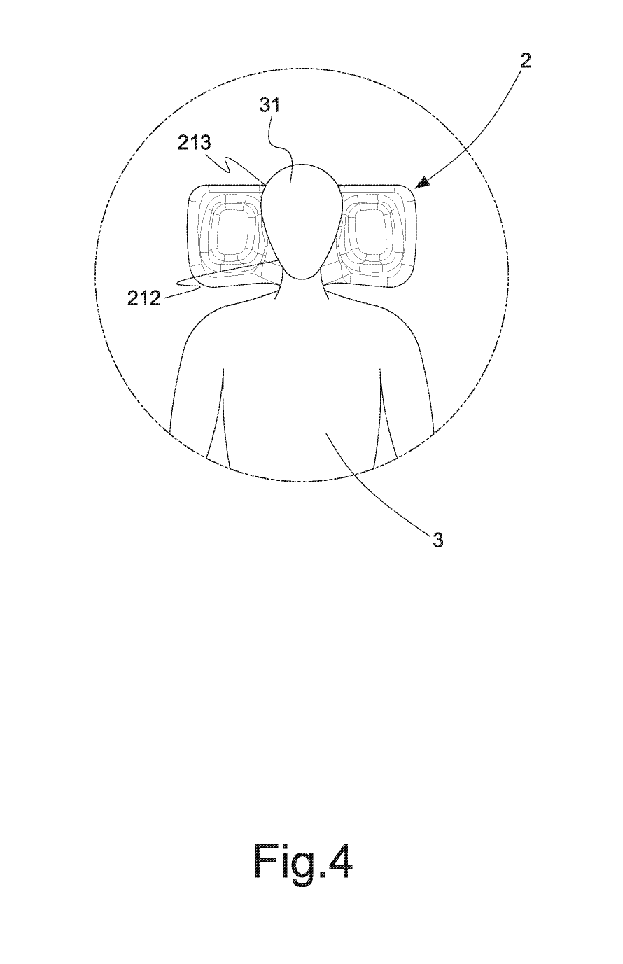

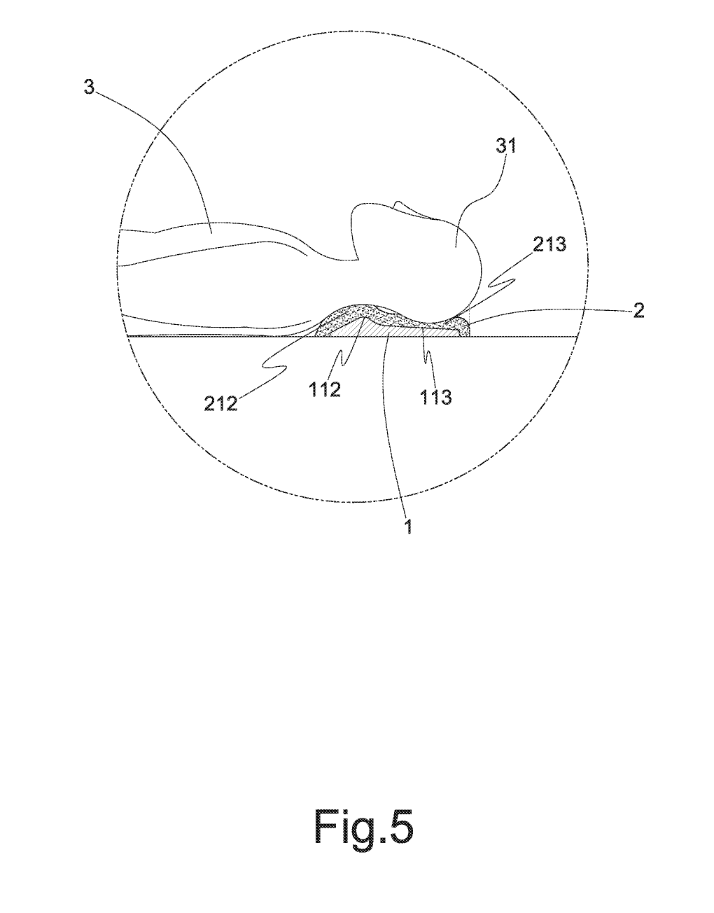

In use, referring to FIG. 2, FIG. 4, and FIG. 5, when a user's body 3 sleep on his back, his head 31 is settled in the central unit 11 of the main body 1. At this time, the raised portion 112 props up the user's cervical vertebra to make his head lean backward, thereby keeping his trachea open and preventing snoring. The inclined portion 111 is configured to receive the neck-back joint of the human body 3. The depressed portion 113 serves to hold and position the afterbrain of the head 31 when the human body 3 is in its sleep-on-back position.

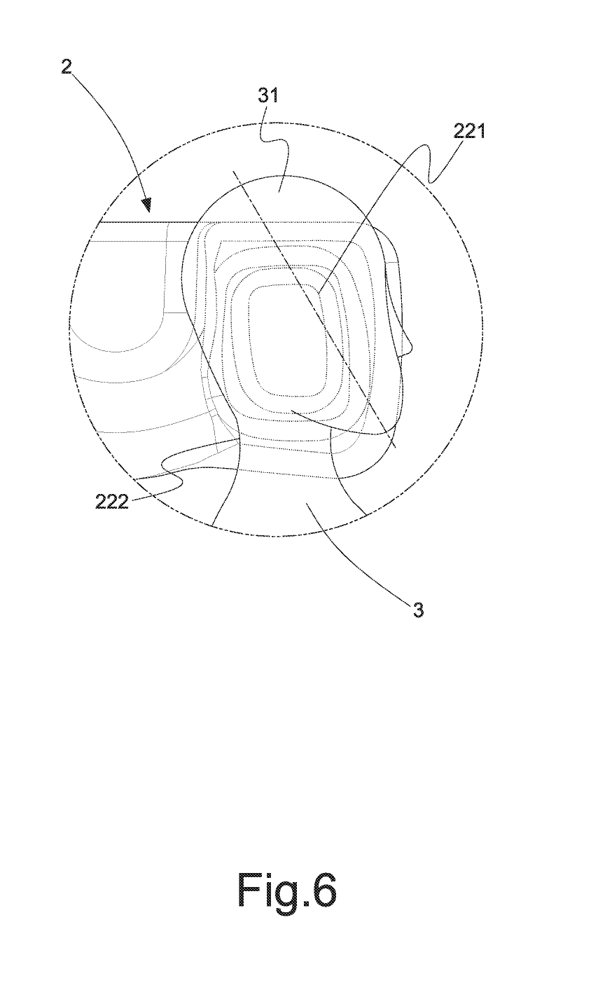

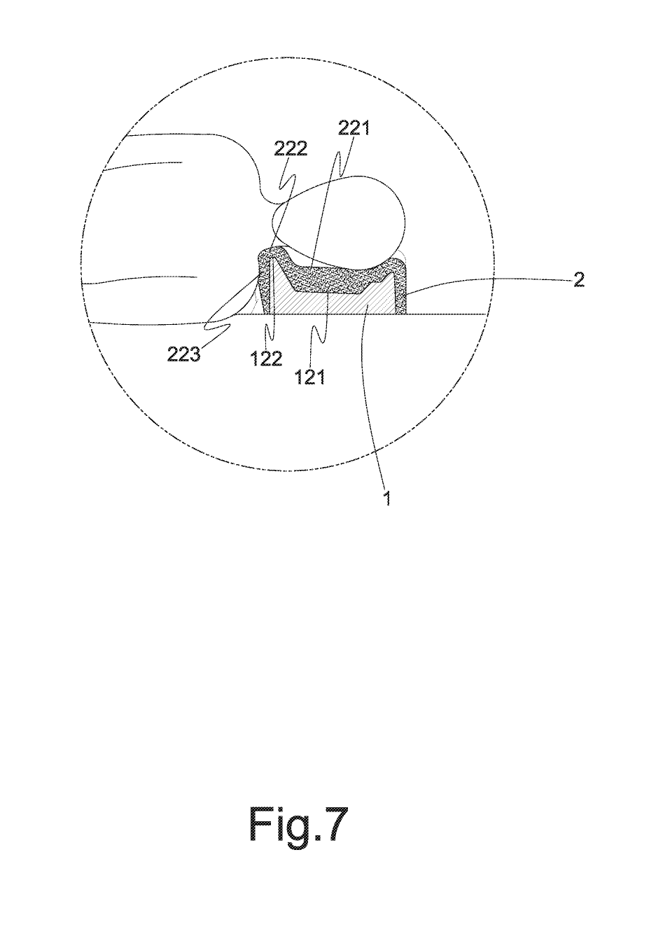

Referring to FIG. 2, FIG. 6, and, FIG. 7, the head 31 of the human body 3 moves to the lateral unit 12 when a user turns his body and becomes sleeping on his side. With the lateral depressed portion 121, combining the lateral frame 122 supporting the user's head 31 from side and the upper lateral depressed portion 221 providing soft touch, the pillow does not press the sleep-on-side user's ear. In addition, the lateral frame 122 provided at the periphery of the lateral depressed portion 121 is so shaped that it guides the head 31 to lean backward for a proper angle, which keeps the user's trachea open and prevents snoring. Moreover, each of the lateral units 12 has its front edge recessed and provided with an inward inclined portion 123, which receives and prevents the shoulder of the human body 3 from compression and discomfort.

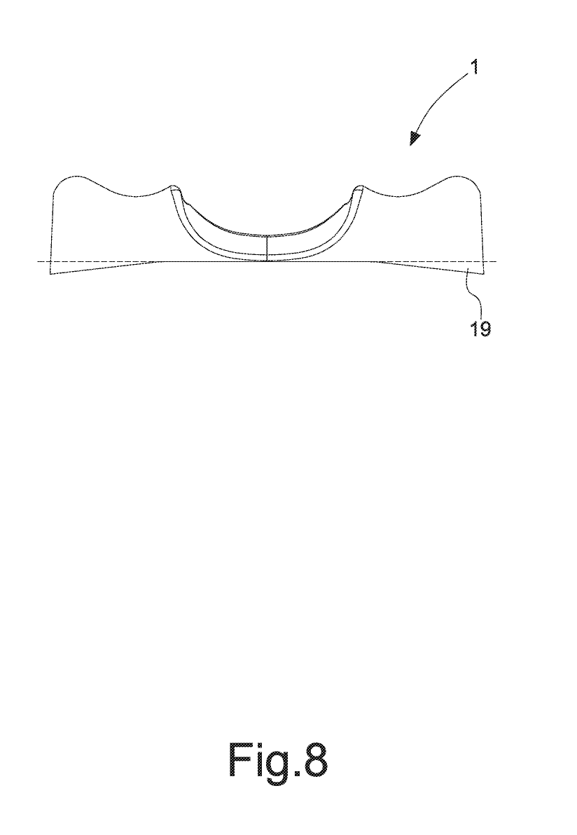

As shown in FIG. 8, in the present invention, the main body has two outward-thickened support members 19 that are provided at two sides of its bottom, respectively. The support members 19 are configured to effectively support the outer sides of the main body, so that even when the pillow bears the weight of the user's head, the overall structure is prevented from slanting outward and deformation. This effectively maintains the pillow's supportive function and keeps a sleep-on-side user's cervical vertebra at a proper angle that allows normal and smooth breath.

To sum up, the disclosed pillow can meet a human body's different requirements for sleep-on-back and sleep-on-side positions in a single structure, and when the user sleeps in both sleep-on-back and sleep-on-side positions it can make the user's head slightly lean backward, so as to keep his trachea open and prevent snoring.

What is claimed is:

1. A pillow, comprising a main body, which is centrally provided with a central unit, the central unit being flanked by two lateral units; the central unit including a raised portion, the raised portion having its rear edge provided with a depressed portion; each said lateral unit being centrally provided with a lateral depressed portion, the lateral depressed portion being peripherally provided with a lateral frame, the lateral frame being such shaped that when a user sleep on his side with the pillow below his head, it makes the user's head lean backward; the pillow also having an upper main body formed on the main body, the upper main body being made of a relatively soft material, and the main body being made of a relatively hard material; the upper main body being shaped according to the main body and comprising an upper central unit and two upper lateral units; each of the upper lateral units formed with an upper lateral depressed portion and an upper lateral frame, the upper lateral depressed portion being located above the lateral depressed portion, and the upper lateral frame being located above the lateral frame.

2. The pillow of claim 1, wherein the raised portion has its front edge adjacent to an inclined portion.

3. The pillow of claim 1, wherein each of the lateral units has its front edge recessed and provided with an inward inclined portion.

4. The pillow of claim 1, wherein the main body has two outward-thickened support members provided at two sides of its bottom, respectively.

5. The pillow of claim 1, wherein the upper central unit is formed with an upper raised portion, an upper inclined portion, and an upper depressed portion, in which each of the upper lateral units has its front edge recessed and provided with an upper inward inclined portion.
